(19) United States
(12) Patent Application Publication
(10) Pub. No.: US 2006/0129038 A1
(43) Pub. Date: Jun. 15, 2006

(54) Optical determination of in vivo properties

(76) Inventors: Alex R. Zelenchuk, Stoughton, MA (US); Howard B. Kaufman, Newton, MA (US)

Correspondence Address: SULLIVAN & WORCESTER LLP, ONE POST OFFICE SQUARE, BOSTON, MA, 02109, US

(21) Appl. No.: 11/109,409

(22) Filed: Apr. 19, 2005

Related U.S. Application Data
(63) Continuation-in-part of application No. 11/011,714, filed on Dec. 14, 2004.

(51) Int. Cl. A61B 5/00

(52) U.S. Cl. 600/322

(57) ABSTRACT

A system and method for determining an in vivo property of a tissue or blood is described. The in vivo property may be a hematocrit value, a hemoglobin concentration, or a combination thereof. The system can automatically determine a location of a subcutaneous blood vessel. Based on the automatically determined location, the system illuminates the blood vessel with a light beam and detects light resulting from the illumination. The system determines the in vivo property based on the detected light. Alternatively, or in combination, the system displays an image corresponding to a spatial relationship between a subcutaneous blood vessel and a light beam. Based on the image, an operator can adjust the light beam with respect to the blood vessel to have a selected spatial relationship. The system determines an in vivo property based on the illumination of the blood vessel when the light beam has the selected spatial relationship.

### RELATED APPLICATIONS

The present application is a continuation-in-part of U.S. application Ser. No. 11/011,714, filed Dec. 14, 2004, which application is incorporated herein by reference in its entirety.

### FIELD OF THE INVENTION

The invention relates to the optical determination of in vivo properties of a solid tissue or blood.

### BACKGROUND

Medical personnel often need to determine properties of human or animal solid tissue or blood. For example, in a diagnostic or surgical setting, one may wish to determine blood hematocrit (Hct), which relates to the abundance of hemoglobin (Hb) and/or red blood cells in blood. Traditional determinations of Hct include drawing blood from a vein and centrifuging the drawn blood to separate cellular and fluid components of the blood.

### SUMMARY OF THE INVENTION

One aspect of the present invention relates to the optical determination of an in vivo property of a tissue or blood and related methods and systems. In various embodiments, the in vivo property is an Hct value, an Hb concentration, or combination thereof. Unless otherwise specified, the in vivo property may be a relative value or an absolute value.

In some embodiments, a method for determining an in vivo blood property includes irradiating a first location of a subject's skin with incident light, detecting exit light resulting from irradiating the first location, at least some of the exit light having passed through a blood vessel of the subject (e.g., a blood vessel having a diameter of at least about 500 microns) and exited the subject's skin at a distance from the first location, irradiating a second location of the subject's skin with incident light, detecting reference light resulting from irradiating the second, different location, at least some of the reference light having passed through at least some subsurface tissue of the subject without passing through the blood vessel, and determining the in vivo blood property based on the first exit light and the reference light.

In some embodiments, the first and second locations of the subjects skin are different.

In some embodiments, the exit light is first exit light and the distance is a first distance and the method further includes detecting second exit light resulting from irradiating the first location, with at least some of the second exit light having passed through a blood vessel of the subject and exited the subject's skin at a second distance from the first location. The first and second distances are typically different. The in vivo blood property is determined based on the first and second exit light and the reference light.

In some embodiments, detecting the first exit light includes detecting light that has exited the skin through a first area of the skin centered about the first distance, detecting the second exit light includes detecting light that has exited the skin through a second area of skin centered about the second distance, and detecting the reference light comprises detecting light that has exited the subject's skin through a third area of the subject's skin that is larger than the first or second areas. Determining the in vivo property includes determining the in vivo property based on the reference light that has exited the skin through the third area of skin. In some embodiments, the third area of the subject's skin is at least about 10 times larger (e.g., at least about 25 times larger) than the first or second areas. In some embodiments, the third area of skin has a lateral dimension of at least about 2 mm. At least one (e.g., both) of the first and second areas of skin may have a maximum lateral dimension of about 0.75 mm or less.

In some embodiments, irradiating the first location and irradiating the second, different location each include irradiating the skin with a beam of incident light having a diameter about the same as or less than a diameter of the blood vessel. For example, the beam of light may have a diameter of about 500 microns or less.

In some embodiments, determining the in vivo blood property based on the first and second exit light and the reference light includes determining the in vivo blood property based on at least: a first portion of the reference light indicative of the total amount of light that has exited the skin through the third area, a second portion of the reference light that is indicative of the total amount of light that has exited the skin through a fourth area of skin centered about the first distance from the second illumination location, and a third portion of the reference light that is indicative of the total amount of light that has exited the skin through a fifth area centered about the second distance from the second illumination location. Typically, the size of the first and fourth areas of skin are about the same and the size of the second and fifth areas of skin are about the same.

In some embodiments, the reference light is detected without the reference light having passed through a blood vessel with a diameter greater than about 100 microns.

In some embodiments, the second distance is at least about twice as large as the first distance. For example, detecting the first light can include detecting light that has exited the subject's skin through an area of the skin that has a maximum dimension smaller than a difference between the first and second distances. In some embodiments, the first distance is at least about 0.5 mm and about 1.75 mm or less and the second distance is at least about 1.5 mm and about 5 mm or less. In some embodiments, the second distance is at least about three times as large as the first distance.

In some embodiments, the method includes detecting second and third reference light resulting from irradiating the first location of the subject's skin with second incident light. The second incident light has a wavelength that is more attenuated by blood than a wavelength of the first incident light. The second reference light is detected after exiting the subject's skin at the first distance from the first location. The third reference light is detected after exiting the subject's skin at the second, different distance from the first location. Determining the in vivo blood property includes determining the in vivo blood property based on the first and second exit light and the first, second, and third reference light.

In some embodiments, determining the in vivo blood property includes determining a difference between the first exit light and the second reference light and a difference between the second exit light and the third reference light.

In some embodiments, a method of determining an in vivo blood property includes detecting first exit light I_{1,1} resulting from irradiating a first location of a subject's skin with incident light, at least some of the first exit light having passed through a blood vessel of the subject and exited the subject's skin at a first distance from the first location, detecting first reference light R_{1,1} resulting from irradiating the first location of the subject's skin with second incident light, at least some of the first reference light having passed through subsurface tissue of the subject and exited the subject's skin at the first distance from the first location, the second incident light having a wavelength that is more attenuated by blood than a wavelength of the first incident light, detecting second reference light I_{2,T} resulting from irradiating a second, different location of the subject's skin with third incident light, at least some of the second reference light having passed through at least some subsurface tissue of the subject without passing through the blood vessel, wherein detecting the second reference light I_{2,T} comprises detecting light that has exited the subject's skin through an area of the subject's skin that is larger than an area through which either the first exit light or first reference light exited the subject's skin, detecting third reference light I_{2,1} resulting from irradiating the second, different location of the subject's skin with incident light, at least some of the third reference light having passed through subsurface tissue of the subject without passing through the blood vessel and exited the subject's skin at the first distance from the second location, and determining the in vivo blood property based on the light I_{1,1}, R_{1,1}, I_{2,T}, I_{2,1}.

In some embodiments, determining the in vivo blood property includes determining a first corrected light intensity I1,C based at least in part on the relationship:

$${I}_{1 , C} = \frac{( {I}_{1 , 1} - {R}_{1 , 1} ) \times {I}_{2 , T}}{{I}_{2 , 1}}$$

In some embodiments, the method includes detecting second exit light I_{1,2} resulting from irradiating the first location of the subject's skin with first incident light, with at least some of the second exit light having passed through the blood vessel of the subject and exited the subject's skin at a second distance from the first location, detecting fourth reference light R1,2 resulting from irradiating the first location of the subject's skin with second incident light, at least some of the second light having passed through subsurface tissue of the subject and exited the subject's skin at the second distance from the first location, detecting fifth reference light I2,2 resulting from irradiating the second, different location of the subject's skin with incident light, at least some of the fifth reference light having passed through subsurface tissue of the subject without passing through the blood vessel and exited the subject's skin at the second distance from the second location, determining the in vivo blood property based on the light I1,1, R1,1, I2,T, I2,1, I1,2, R1,2, I2,2.

In some embodiments, the method includes determining a first corrected light intensity I_{1,C} based at least in part on the relationship:

$${I}_{1 , C} = \frac{( {I}_{1 , 2} - {R}_{1 , 2} ) \times {I}_{2 , T}^{5}}{{I}_{2 , 2}}$$

and determining a second corrected light intensity I_{2,C} based at least in part on the relationship

$${I}_{2 , C} = \frac{( {I}_{1 , 1} - {R}_{1 , 1} ) \times {I}_{2 , T}^{10}}{{I}_{2 , 1}}$$

In some embodiments, a method for determining an in vivo blood property includes automatically determining a location of a blood vessel of a subject, illuminating the blood vessel with light by illuminating a first location of skin of the subject with incident light, detecting exit light resulting from illuminating the first location of skin, illuminating a second location of the skin of the subject with incident light, the second location being spaced apart from the first location, detecting reference light resulting from illuminating the second location of skin, at least some of the second exit light having passed through at least some sub-surface tissue of the subject without passing through the blood vessel, and determining an in vivo blood property based on the exit light and the reference light.

In some embodiments, detecting the reference light includes detecting light that has exited the subject's skin through an area of the skin that is larger than an area of the skin through which the exit light exited and determining the in vivo property includes determining the in vivo property based on the reference light that has exited through the area of the subjects skin that is larger than the area of skin through which exit light exited the skin.

In some embodiments, the exit light is first exit light and the method includes detecting second exit light resulting from illuminating the first location of skin. The second exit light typically exits the skin at a different distance from the first location than the first exit light. The in vivo blood property is determined based on the first and second exit light and the reference light.

In another embodiment, a system for determining an in vivo blood property includes a light source configured to irradiate first and second spaced-apart locations of a subject's skin with incident light, a detector configured to detect exit light resulting from irradiating the first location, at least some of the exit light having passed through a blood vessel of the subject and exited the subject's skin at a distance from the first location and detect reference light resulting from irradiating the second, different location, at least some of the reference light having passed through at least some subsurface tissue of the subject without passing through the blood vessel, and a processor configured to determine the in vivo blood property based on the first exit light and the reference light. In some embodiments, the light source is configured to irradiate each of the first and second locations with a beam of incident light having a diameter of about 2 mm or less.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entireties. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE FIGURES

FIG. 1 is a schematic of a system for determining an in vivo property of a solid tissue or blood of a human or animal. The system is shown ready for an exemplary determination with a sensor module positioned to illuminate a wrist of a human subject with light and to detect light resulting from the illumination.

FIG. 2 is a schematic representation of an exemplary spatial relationship between a light beam projected by the sensor module of the system of FIG. 1 and a blood vessel of the wrist.

FIG. 3 is a cross-sectional side view of the sensor module of the system of FIG. 1 positioned as shown in FIG. 1. The sensor module projects a light beam, which passes through the skin and illuminates a blood vessel of the wrist.

FIG. 4A is a graph that illustrates the change in intensity with distance for light propagating within the blood vessel away from a light beam projected by the sensor module as shown in FIG. 3.

FIG. 4B is a top perspective view of skin illuminated by the system of FIG. 1.

FIG. 4C is a plot showing the change in the ratio of the intensity for light detected at different distances from an illuminating light beam.

FIG. 5 is a cross-sectional side view of a light beam illuminating skin and subcutaneous tissue to determine a contribution of subcutaneous tissue to light detected with the system of FIG. 1.

FIG. 6A is a cross-sectional side view of a light beam illuminating a location of skin offset from a blood vessel.

FIG. 6B is a top perspective view of the illumination of FIG. 6A.

FIG. 7 illustrates a family of Hct curves each determined from intensity measurements obtained from each of multiple reference subjects having about the same Hct.

FIG. 8 is a schematic of a probe for determining a contribution from skin and certain subcutaneous tissues to measurements made with the system of FIG. 1.

FIG. 9 is a schematic of an optical face of the sensor module of the system of FIG. 1. The optical face includes a first set of terminal optical fiber ends arranged to project a pattern of light beams onto the skin of the wrist and a set of optical fiber entrances arranged to transmit light received by the optical face to a detector.

FIG. 10 is a representation of a pattern of light beams projected onto the wrist by the sensor module of the system positioned as shown in FIG. 1. The inset illustrates a spatial relationship between light beams of the projected pattern and several blood vessels.

FIG. 11 is a side view of another sensor module.

FIG. 12 is schematic diagram of an optical face of a sensor module. A plurality of light sources surround the optical face.

FIG. 13 is a representation of an integrated system for determining an in vivo property of a tissue of a human or animal.

FIG. 14 is a representation of a system for performing an injection and/or marking an injection site.

### DETAILED DESCRIPTION

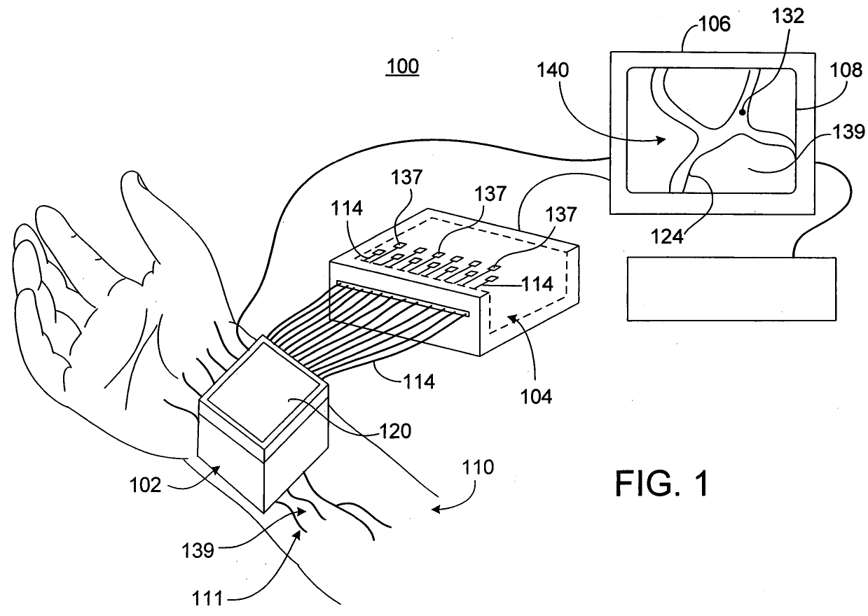

Referring to FIGS. 1-3, a system 100 is configured to determine at least one in vivo property of a tissue or blood of a mammal (e.g., a human subject). In some embodiments, the system determines a Hematocrit (Hct) value and/or related property of the subject's blood. For example, the Hct value of blood can be determined as the percentage of total blood volume occupied by red blood cells, which is proportional to the hemoglobin (Hb) concentration of the blood.

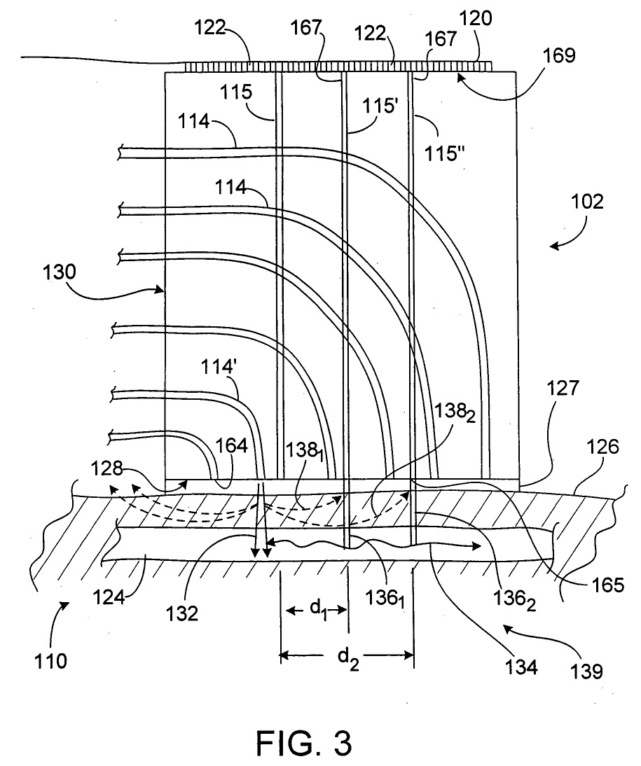

System 100 includes a sensor module 102, a light source 104, a processor 106, and a display 108. Sensor module 102 includes first and second pluralities of optical fibers 114, 115 and a multidimensional detector 120 (e.g., a charge coupled device (CCD) or charge injection detector (CID)) having a plurality of pixels 122 (FIG. 3). Each fiber 114 of the first plurality of optical fibers can project light from light source 104 as a light beam from an optical face 128 of the sensor module. Fibers 115 of the second plurality of optical fibers transmit light received by optical face 128 to various pixels 122 of detector 120. Processor 106 determines an in vivo blood property (e.g., an Hb concentration and/or an Hct value) based on light detected by the pixels. Display 108 can display an image 140 of the detected light.

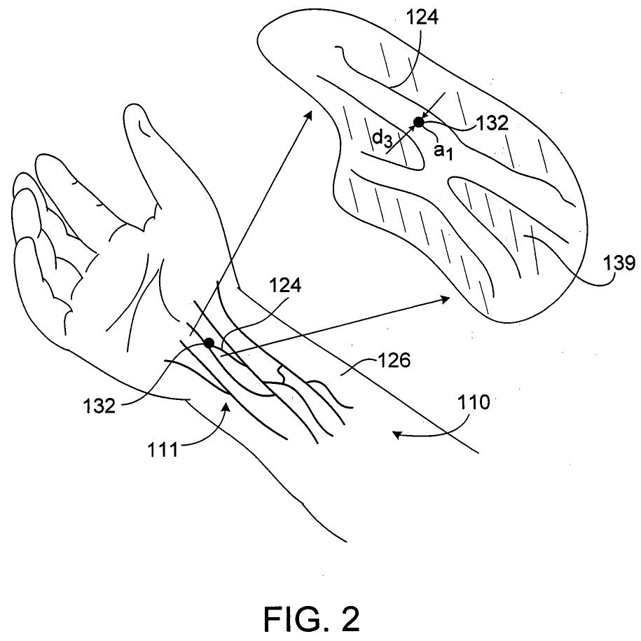

In an exemplary use of system 100, an operator positions optical face 128 of the sensor module 102 generally adjacent a human wrist 110 (FIG. 3), which includes blood vessels of network 111 within surrounding subcutaneous tissue 139 (FIG. 2). Blood vessels of network 111 are typically larger than blood vessels that might be within subcutaneous tissue 139. For example, one or more vessels of network 111 typically have a diameter of at least about 750 microns (e.g., at least about 1000 microns, at least about 1250 microns, at least about 1500 microns, at least about 2000 microns). Surrounding subcutaneous tissue 139 typically contains no vessels larger than about 200 microns (e.g., no vessels larger than about 150 microns, no vessels larger than about 100 microns, no vessels larger than about 75 microns) in diameter.

System 100 illuminates skin 126 of the wrist (e.g., with light beams projected from many fibers 114) and detects light that interacts with (e.g., is reflected and/or scattered from) blood vessel network 111 and subcutaneous tissue 139. Display 108 displays an image 140 (FIG. 1) of vessel network 111 and subcutaneous tissue 139. In the image 140, blood vessels appears darker than surrounding subcutaneous tissue 139 because the blood vessels absorb the illuminating light more strongly than the surrounding subcutaneous tissue.

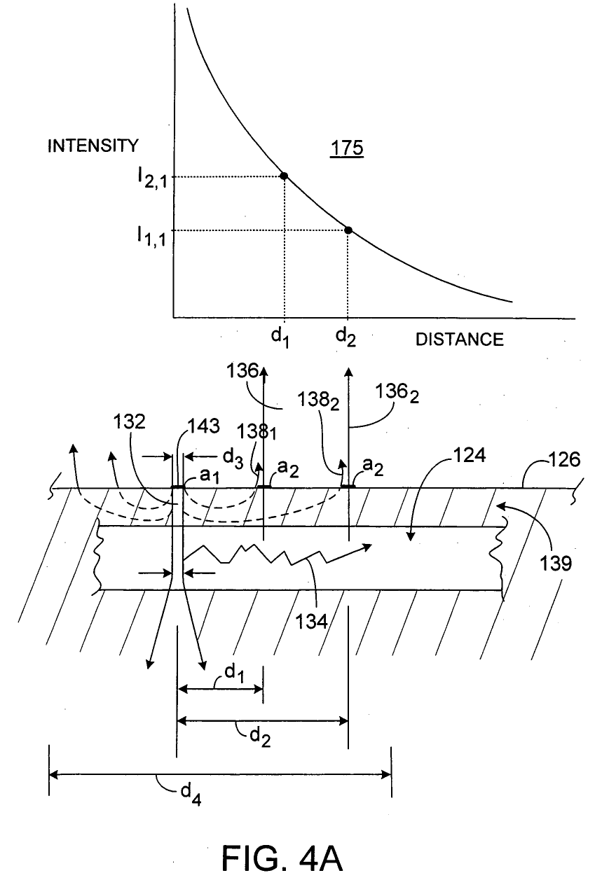

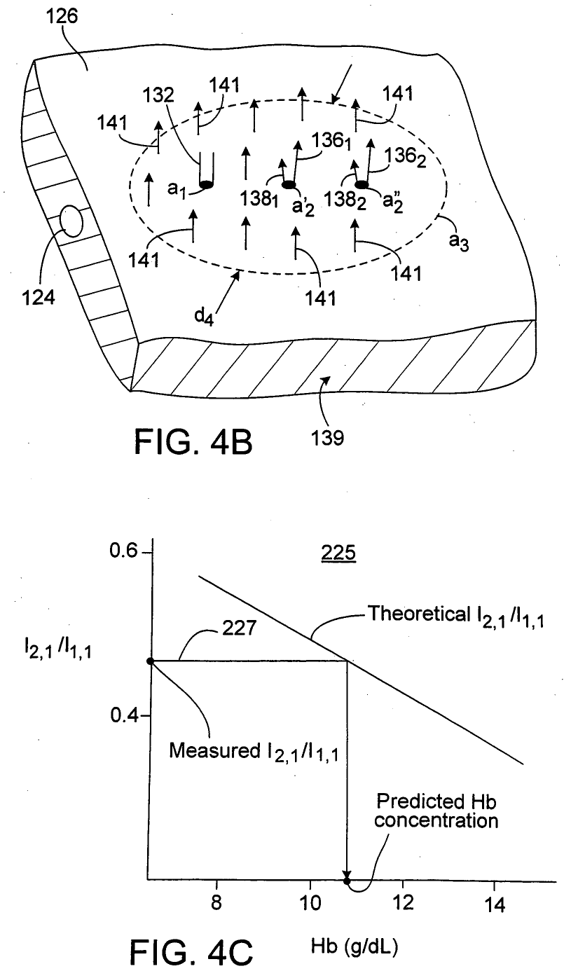

Based on image 140, the operator positions sensor module 102 (e.g., by moving sensor module 102 with respect to the wrist 110) so that a light beam projected from a selected fiber (e.g., light beam 132 projected from fiber 114′) illuminates a first illumination location 143 of skin 126 (FIG. 4B). In general, the orientation of beam 132 and the position of location 143 are arranged so that at least some of the light enters a blood vessel of network 111 (e.g., a blood vessel 124). Typically, beam 132 is oriented perpendicular to skin 126 and first illumination location 143 overlies blood vessel 124.

Area a₁ of first illumination location 143 is determined by the size (e.g., diameter d₃) of light beam 132 (FIG. 4A). For example, each optical fiber 114 may project a beam having a diameter d3 (FWHM) of 2.5 mm or less (e.g., about 1.75 mm or less, about 1.25 mm or less, about 0.75 mm or less, about 0.5 mm or less) within about 4 mm from optical face 128. In some embodiments, area a₁ is about 5 mm² or less (e.g., about 3 mm² or less, about 2 mm² or less, about 0.75 mm² or less, about 0.35 mm² or less, about 0.2 mm² or less).

At least a portion 134 of the light of beam 132 enters blood vessel 124 and propagates therein (e.g., generally along the longitudinal axis of the blood vessel) interacting with blood components (e.g., by absorption, scattering, and/or reflection from red blood cells) (FIGS. 3 and 4a). Scattering, reflection and/or other processes direct at least some of light 134 back out of blood vessel 124 and through skin 126. As examples, light 136₁ exits through skin 126 at a distance d₁ from first illumination location 143 and light 136₂ exits through skin 126 at a greater distance d₂ from first illumination location 143.

Because beam 132 tends to spread out after passing through skin 126, another portion of the beam propagates within subcutaneous tissue 139 and exits from skin 126 without having entered vessel 124 (FIGS. 3 and 4A). As examples, light 138₁ passes through subcutaneous tissue 139 and exits from skin 126 at distance d1 from light beam 132 and light 138₂ passes through subcutaneous tissue 139 and exits from skin 126 at distance d₂ from first illumination location 143.

In general, the difference between distances d₁ and d₂ is greater than diameter d₃ of light beam 132.

At least some of the exiting light is received by one or more fibers 115, which carry the light to pixels 122 of detector 120 (FIG. 3). For example, fiber 115′ receives light exiting through an area a′₂ centered distance d₁ from first illumination location 143 and fiber 115″ receives light exiting through an area a″₂ centered distance d₂ from first illumination location 143. Hence, fiber 115′ transmits light 136₁ and 138₁ to pixels of detector 120 and fiber 115″ transmits light 136₂ and 138₂ to other pixels of detector 120.

The intensity of light detected after exiting the skin is I_{i,j}, where the skin irradiation location varies with index I and the distance from the skin irradiation location varies with index j. For example, the total intensity detected after exiting through area a′₂ centered about first distance d1 from the first illumination location 143 is I_{1,1} (e.g., 136₁ plus 138₁). The total intensity detected after exiting through area a″2 centered about second distance d1 from the first illumination location 143 is I_{2,1} (e.g., 136₂ plus 138₂) (FIG. 4B).

In some embodiments, areas a′₂ and a″₂ are at least about 0.5 mm² (e.g., at least about 0.75 mm², at least about 1.25 mm², at least about 2 mm²). In some embodiments, areas a′₂ and a″₂ are about 5 mm² or less (e.g., about 4 mm² or less, about 3 mm² or less, about 2 mm² or less). Typically, areas a′₂ and a″₂ are the same, although one of these areas (e.g., area a″₂) may be larger than the other.

Typically, all of the light from beam 132 that exits from skin 126 does so within an area a₃ surrounding first illumination location 143 (FIG. 4B). Area a₃ is typically between about * mm² and about *mm² (e.g., between about *mm² and about *mm²). A diameter d₄ of area a₃ is typically at least about 3 mm (e.g., at least about 4 mm, at least about 5 mm, at least about 6 mm). Diameter d₄ is typically about 15 mm or less (e.g., about 12.5 mm or less, about 10 mm or less, about 7.5 mm or less).

The intensity of light exiting from skin 126 depends upon the original illumination intensity and the distance traveled beneath the skin. In general, the farther light travels within the blood vessel, the lower its intensity upon exiting from skin 126. For example, as seen in FIG. 4A, intensity I_{1,1} is larger than intensity I_{2,1} because of light 136₂ and 138₂ traveled a farther beneath skin 126 than light 136₁ and 138₁.

The intensity of light exiting from skin 126 also depends upon in vivo blood properties (e.g., the Hb concentration and/or Hct value). For example, FIG. 4C shows that the ratio of the intensities I_{2,1}/I_{1,1} decreases with increasing hemoglobin (Hb) concentration (e.g., with increasing Hct). In general, light that has passed within blood vessel 124 (e.g., light 136₁ and 136₂) is more sensitive to in vivo blood properties than light that has passed only within subcutaneous tissue (e.g., light 138₁ and 138₂).

Because of the sensitivity of light 136₁ and 1362 to in vivo blood properties, detected intensities I_{1,1} and I_{2,1} or a function of these intensities (e.g., the ratio of intensities I_{1,1} and I_{2,1}) can be used to determine the Hb concentration and/or Hct. For example, the detected intensities I_{1,1} and I_{2,1} or function of these intensities can be compared to theoretically predicted values (e.g., using a photon diffusion model) to predict the in vivo blood property. For example, line 227 of plot 225 in FIG. 4C illustrates how the hemoglobin concentration can be predicted using a ratio of measured intensities I_{1,1} and I_{2,1} and a theoretical model of these intensities.

Typical theoretical models include one or more parameters such as the wavelength of the illuminating light beam, the scattering and absorption cross-sections of red blood cells and other blood components at the illuminating light wavelength, the scattering and absorption cross-sections of subcutaneous tissue 139, and distances d₁ and d₂. Theoretical models and parameters useful for such models are discussed in, e.g., Reynolds, L. O., Optical Diffuse Reflectance and Transmittance From An Anisotropically Scattering Finite Blood Medium, Ph.D. Thesis, Dept. Electrical Eng., Univ. of Wash., 1975; Reynolds, L. O. et al. Diffuse Reflectance From A Finite Blood Medium: Applications To The Modeling Of Fiber Optic Catheters, Applied Optics, 15(9), 2059-2067, 1967; and Bohren, C. F. et al., Absorption and Scattering of Light by Small Particles, New York, Wiley & Sons, 477-482, 1983, each of which documents is incorporated herein by reference.

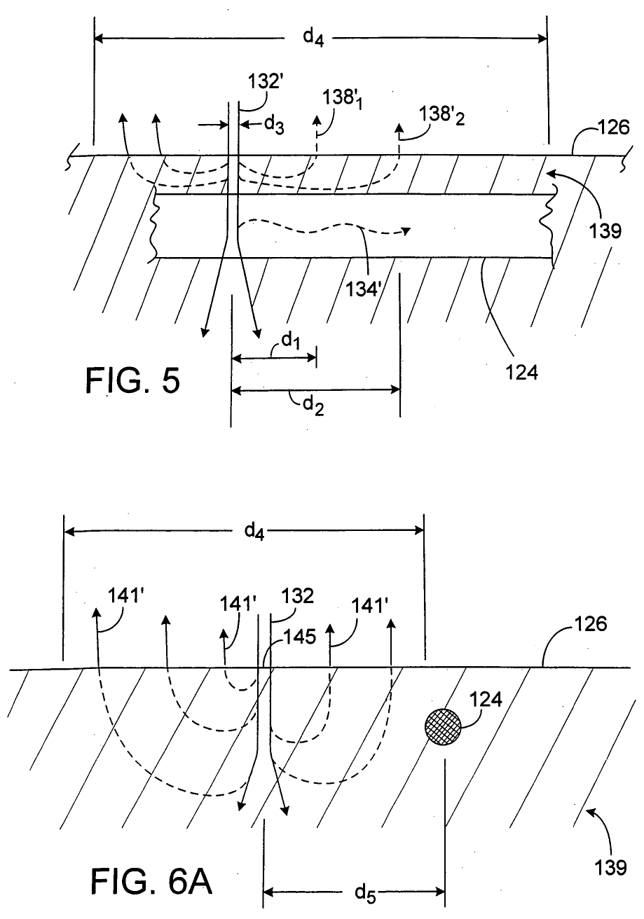

In some embodiments, system 100 is configured to determine the relative intensity of light that has passed only within subcutaneous tissue 139 and not within vessel 124 (e.g., light 138₁ and 138₂) and correct the detected intensities I_{1,1} and I_{2,1} for the presence of this light. For example, referring to FIG. 5, system 100 determines a relative contribution of light that has passed only within subcutaneous tissue 139 and not within vessel 124 by illuminating first illumination location 143 with a light beam 132′ having a wavelength that is more absorbed by blood than the wavelength of light beam 132. In some embodiments, the wavelength of beam 132′ is less than about 700 nm (e.g., between about 550 and 650 nanometers).

Light 134′ of light beam 132′ that enters vessel 124 is absorbed by the blood and little or none exits from skin 126. On the other hand, at least some light from light beam 132′ exits from skin 126 after passing only through subcutaneous tissue 139, which contains substantially less blood than vessel 124. As examples, light 138′₁ passes through subcutaneous tissue 139 and exits with an intensity R_{1,1,} through area a′₂ of skin 126 centered about distance d₁ from light beam 132′ and light 138′₂ passes through subcutaneous tissue 139 and exits with an intensity R_{2,1} through area a″₂ of skin 126 centered about distance d₂ from light beam 132′. Because the amount of absorption by subcutaneous layer 139 is less dependant on the wavelength of the illuminating light beam, the intensity R_{1,1,} of light 138′₁ corresponds generally to the intensity of light 138₁ and the intensity R_{2,1} of light 138′₂ corresponds generally to the intensity of light 138₂ (FIG. 4A). Consequently, the intensities R_{1,1} and R_{2,1} can be used to correct the intensities I_{1,1} and I_{2,1} for the presence of light 138₁ and 138₂. For example, intensities I_{1,1,} and I_{2,1} can be corrected by respectively subtracting intensities R_{1,1} and R_{2,1}. In general, the corrected intensities (e.g., I_{1,1}-R_{1,1} and I_{2,1}-R_{2,1}) are more sensitive to in vivo blood properties than the detected intensities (e.g., I_{1,1} and I_{2,1}) and can be used (e.g., by comparison to a theoretical model as discussed above) to predict an in vivo blood property.

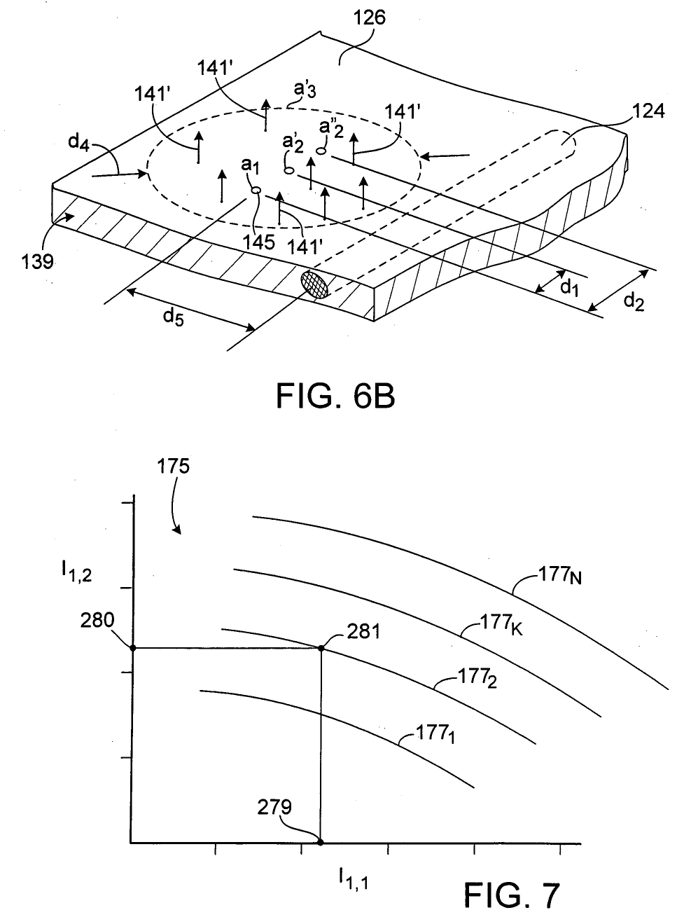

Turning now to FIGS. 6A and 6B, another example of correcting detected intensities I_{1,1} and I_{2,1} includes normalizing these intensities by the intensity of light that exits through areas a′2 and a″2 relative to the total intensity that exits from the skin. Typically, determining the total intensity of exiting light includes using an illumination beam 132″ to illuminate a second illumination location 145 offset from first illumination location 143 by a distance d₅. Light from beam 132″ passes through skin 126 and into subcutaneous tissue 139 before exiting from the skin as light 141′ within an area a′₃. Typically, all of the light resulting from beam 132″ that exits from skin 126 does so within area a′₃, which typically has about the same dimensions as area a₃ discussed above. System 100 collects light exiting within area a′3 (e.g., using a plurality of fibers 115) to estimate the total intensity I_{2,T} of exiting light 141′. A portion of light 141′ exits with intensity I_{2,1} from skin 126 through an area a′₂ located distance d₁ from second illumination location 145. A portion of light 141′ exits with intensity I_{2,2} from skin 126 through an area a′₂ located distance d₂ from second illumination location 145.

Intensities I_{2,T} and _{2,1} can be used to correct intensity I_{1,1} according to:

$${I}_{1 , C 1} = \frac{( {I}_{1 , 1} - {R}_{1 , 1} ) \times {I}_{2 , T}}{{I}_{2 , 1}}$$

and intensities I_{2,T} and I_{2,2} can be used to correct intensity I_{1,2} according to:

$${I}_{1 , C 2} = \frac{( {I}_{1 , 2} - {R}_{1 , 2} ) \times {I}_{2 , T}}{{I}_{2 , 2}}$$

where corrected intensities I_{1,C1} and I_{2,C1} respectively correspond to I_{1,1} and I_{2,1} and can be used (e.g., by comparison to a theoretical model as discussed above) to predict an in vivo blood property. Determining the total intensity of exiting light offset from the blood vessel allows the contribution of the non-blood vessel subcutaneous tissue to be determined. Beam 132″ typically has the same properties (e.g., wavelength and/or size) as beam 132.

While determination of in vivo blood properties has been described based on the comparison of one or more detected intensities to a theoretical model, other methods can be used. For example, one or more detected intensities can be compared to experimental values (e.g., intensity values detected or determined from one or more reference subjects). In some embodiments, one or more detected intensities (e.g., corrected intensities I_{1,C1} and I_{2,C1}) are compared to one or more intensity values detected from each of multiple reference subjects having a known Hct value. The known intensity values can be determined as desired (e.g., by using an in vitro blood analysis method).

Typically intensity values (e.g., corrected intensities I_{1,C1} and I_{2,C1}) from reference subjects having about the same Hct value are grouped together (e.g., by averaging). FIG. 7 shows a family 175 of curves 177ₖ, where each curve is the average of intensity values detected from multiple reference subjects each having blood of about the same Hct value. Such curves can be stored, for example, as a look-up table.

In use, one or more detected intensity values (e.g., corrected intensities I_{1,C1} and I_{2,C1}) are detected or determined from a subject whose Hct is to be determined. The intensity value(s) are compared to the intensity values from the multiple reference subjects to determine the instant subject's Hct value or other in vivo blood property. For example, FIG. 7 shows that detected intensities 279 and 280 correspond to a point 281 falling on Hct curve 177₂. Any two detected intensities that correspond to a point on Hct curve 177₂, would indicate the same Hct value.

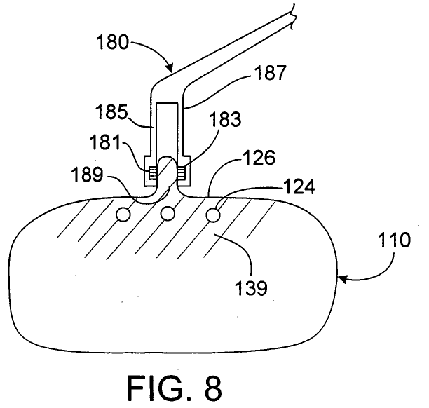

In some embodiments, system 100 is configured to measure or determine the extent to which skin 126 attenuates the illuminating light beam. Referring to FIG. 8, for example, system 100 can include a probe 180 having first and second probe arms 185, 187 respectively having a light source 181 and a detector 183. Probe 180 detects light transmitted through a flap 189 of skin 126 (e.g., skin of the subject's wrist 110). Flap 189 contains little or no subcutaneous tissue 139. Probe 180 generates a detector signal from the detected light. Based on the detector signal, processor 106 can determine a contribution of the skin 126 and underlying subcutaneous tissue to measurements made with sensor module 102. The light source need not emit light at the same wavelength as light used to determine the in vivo blood property.

While probe 180 is described as having a light source and detector on different sides of flap 189, other configurations can be used. For example, the light source and detector can be spaced apart from one another within the same probe arm on one side of flap 189. The detector detects light reflected by skin 189 and any subcutaneous tissue present within flap 189. In some embodiments, the probe arm opposite the light source includes a material that prevents light that reaches the opposite probe arm from reentering the skin and being detected. In some embodiments, the opposite probe arm includes a material having optical properties indicative of a response of blood having a particular Hct or Hb. For example, in some embodiments, the material is a polymer (e.g., a plastic) pigmented to correspond with blood having a particular Hct or Hb.

While first and second illumination locations 143, 145 have been described as different, the locations may overlap or be identical. For example, in some embodiments, the angle of incidence with respect to the skin of the illumination beam is changed so that the beam illuminates vessel 124 at a first angle of incidence (e.g., so that light that passes along the vessel can be detected) and does not substantially illuminate vessel 124 at a second angle of incidence (e.g., so that light that has not passed along the vessel can be detected.

Referring back to FIG. 1, components of system 100 are now discussed in further detail. Light source 104 provides light having a wavelength suitable for determining a location of a blood vessel and/or for determining an in vivo property of blood or tissue. Exemplary light sources include lamps, e.g., incandescent sources, and solid-state sources, e.g., light emitting diodes or diode lasers. The light source may emit light in the visible (e.g., with a wavelength of from about 630 to about 670 nm), near infrared (e.g., with a wavelength of from about 670 to about 1000 nm), or infrared (e.g., with a wavelength of from about 1000 nm and about 1500 nm). In some embodiments, the light source emits light having a narrow bandwidth, e.g., less than about 25 nm at full width half maximum (FWHM). The emitted light may be centered about a selected wavelength, e.g., about 802 nm, about 820 nm, or about 880 nm. In various embodiments, the narrow band light has a wavelength centered about an isobestic point of blood. For example, the light may have a wavelength that corresponds to the isobestic point of oxygenated and de-oxygenated hemoglobin forms.

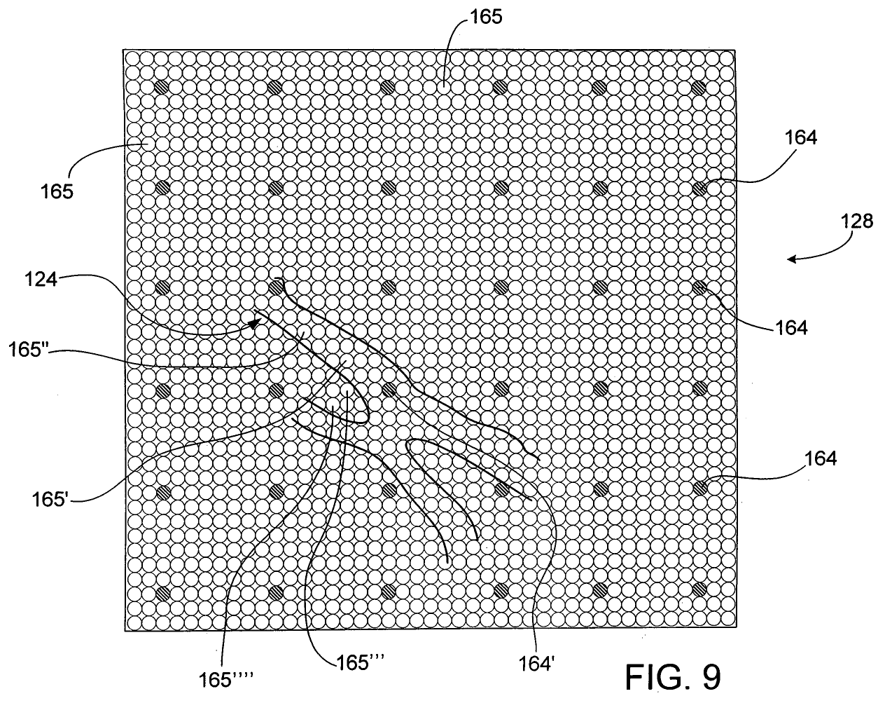

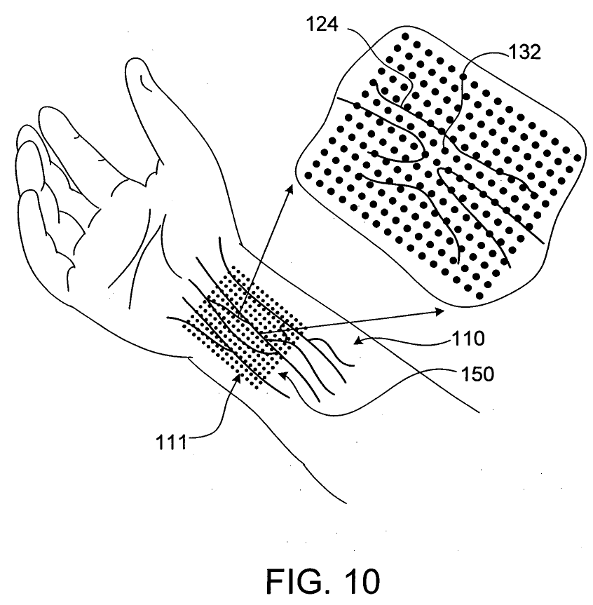

Referring also to FIGS. 9 and 10, sensor module 102 projects light from the light source as a pattern 150 of discrete light beams onto the subject. Light is transmitted from the light source to the optical face of the sensor module by optical fibers 114, each of which terminates at a respective terminal end 164. The terminal ends 164 are arranged in a pattern of rows and columns about the optical face 128. The pattern 150 of projected light beams corresponds to the pattern of terminal ends 164.

As seen in FIG. 3, optical fibers 114 enter sensor module 102 via a side 130 and traverse an arcuate path to reach optical face 128. The optical fibers forming terminal ends 164 along a given row are aligned vertically to limit the area obscured by the fibers.

Although FIGS. 3 and 9 illustrate a 6×6 pattern of terminal ends 164, sensor module 102 can include more or fewer terminal ends 164. Embodiments of sensor module 102 include a sufficient number of terminal ends 164 such that when sensor module 102 is positioned adjacent an adult human wrist at least one of the ends projects a light beam to illuminate blood vessel 124. Embodiments of sensor module 102 may include at least 20, at least 50, at least 75, or at least 100 terminal ends 164 at optical face 128. The terminal ends of the optical face 128 may be arranged over an area of about 5 cm² (e.g., about 8 cm², about 15 cm², about 20 cm²). The pattern of terminal ends may include a varying density of ends 164. In various embodiments, the density variation corresponds to the distribution of vessels within network 111, with the greatest density of terminal ends corresponding generally with the pattern of blood vessels of a subcutaneous region, e.g., of the human wrist.

In FIG. 3, a coupling element 127 is disposed between the optical face 128 and skin 126. Coupling element 127 can include, e.g., a gel, a viscous liquid, or polymer sheet to reduce scattering that might occur at the air-skin interface and air-optical face interface.

The light beam projected by each fiber 114 can be have various shapes including circular, square, or elongated in at least one dimension. In such embodiments, the light beam may have a minor dimension having a width (FWHM) corresponding to light beam diameter d₃.

System 100 can be configured so that terminal ends 164 project light beams subjectly, simultaneously, sequentially, or in subsets of less than all the terminal ends. For example, each fiber 114 is coupled to a respective light emitting diode 137. Processor 106 operates some or all of the diodes independently of the others to project any combination of light beams from terminal ends 164 of optical face 128.

In alternative embodiments, light source 104 includes only one or a few light sources, each coupled to more than one fiber 114. The terminal ends 164 of the fibers 114 coupled to any one light source can be spaced apart at optical face 128 so that detected light resulting from the illumination by each optical fiber 114 can be distinguished from detected light resulting from illumination by other optical fibers 114. Embodiments can include micro-actuated mirrors, shutters, liquid crystal filters, or the like to selectively couple light to one or more selected fibers 114 associated with a single light source.

Sensor module 102 includes a plurality of light guiding elements 115 (only two of which are shown in FIG. 3) to guide light received by different locations of optical face 128 to different pixels 122 of detector 120. Each light guiding element 115 has an entrance aperture 165 at the optical face 128 and a terminal end 167 located at an opposite face 169 of the sensor module. Each of a plurality of terminal ends 167 (e.g., all of the terminal ends) are optically coupled to at least one pixel 122 of detector 120. Each of a plurality of pixels 122 (e.g., all of the pixels) are optically coupled to at least one terminal end 167. Hence, sensor module 102 can obtain an image of subcutaneous features without a lens or other optic with focusing power. In various embodiments, light guiding elements 115 include a plurality of waveguides, a plurality of optical fibers, one or more optics with focusing power, e.g., one or more lenses or mirrors, or combination thereof. Sensor module 102 can include a beam splitting optic to direct light toward the subject yet allow a portion of light exiting the skin to pass through the beam splitting optic to detector 120.

Returning to FIG. 9, an exemplary spatial relationship between a given terminal end 164′, blood vessel 124, and entrances 165′, 165″ to two different optical fibers 115 is illustrated. Upon determination of the location of blood vessel 164′, system 100 illuminates the blood vessel 124 via a light beam projected from the terminal end 164′ of a fiber 114. Light resulting from the illumination and exiting the skin can be received by any of the fiber entrances 165 and detected by detector 120. Light received by fiber entrances 165′ and 165″, however, has passed respective, different distances within blood vessel 124. An in vivo blood property can be determined based upon the light intensity detected by pixels 122 coupled to light guiding elements 115 extending from entrances 165′ and 165″. On the other hand, light received by entrances 165′″ and 165″″ will have passed approximately the same distance within vessel 124 before passing out of the blood vessel and into the surrounding subcutaneous media, e.g., tissue. Based on the spatial relationship between the vessel 124 and the projected light beam, processor 106 can select the light guiding elements 115 that will be used to collect light for determining the in vivo blood property. For example, processor 106 may select fibers that intersect the blood vessel at longitudinally or axially aligned locations with respect to the illuminating light beam.

In various embodiments, sensor module 102 includes a sufficient number of light guiding elements 115 and pixels 122 to provide optical data with a resolution sufficient to allow an operator to adjust the position of a light beam with respect to a subcutaneous blood vessel and/or to allow processor 106 to automatically determine the location of a blood vessel based on the optical data. Sensor module 102 can include at least 1, 50, 250, 1000, 2500, or more light guiding elements 115. In various embodiments, the centers of adjacent fiber entrances 165 are spaced apart along at least one dimension by less than about 250, 125, 75, 25 μm, or less.

As shown in FIG. 1, optical data from detector 120 can be displayed as image 140 including one or more blood vessels of network 111. In some embodiments, the image 140 may not include an image of some or all light beams projected from terminal ends 164 because the fibers 114 extending within the sensor module can block light from reaching detector 120. Nonetheless, an operator or processor 106 can determine whether a given terminal end 164 is aligned with a blood vessel based on light received by fibers 115 in the vicinity of the given fiber 114. Such a condition exemplifies that optical data output by the sensor module need not expressly include a light beam to be indicative of a spatial relationship between the light beam and a blood vessel.

In some embodiments, system 100 assists an operator in positioning light beams 132, 132′, and 132″. For example, processor 106 can automatically determine a location of blood vessel 124 (e.g., determine the location of vessel 124 relative to sensor module 102) and operate system 100 to illuminate the blood vessel with light beam 132 (FIGS. 2 and 3). Typically, sensor module 102 obtains optical data, whether digital or analog, from the subcutaneous network 111 of blood vessels and surrounding tissue 139. Processor 106 processes the optical data of the wrist to locate regions that correspond to one or more blood vessels of network 111. Such determined locations may be relative, e.g., relative to some portion of sensor module 102 or to the light beam 132.

In various embodiments, processor 106 receives optical data from detector 120. Processor 106 distinguishes blood vessels from the surrounding subcutaneous media based on properties of the detected light, e.g., the intensity and varying contrast of the detected light. For example, processor 106 may subject the optical data to segmentation, e.g., by threshold techniques, edge-based methods, region-based techniques, or connectivity-preserving relaxation techniques. Processor 106 may determine boundaries between vessels and surrounding media, such as by use of continuous edges and/or allowable bifurcation patterns of network 111. The optical data may be subjected to edge and/or contrast enhancement to better differentiate vessels from surrounding media. Once one or more vessels have been located, e.g., with respect to a portion of sensor module 102, processor 106 selects an appropriate fiber 114 with which to illuminate the vessel.

System 100 performs one or more different actions upon determining the location of the one or more blood vessels and/or subcutaneous tissue 139 depending, for example, on whether illumination beams 132, 132′, or 132″ are being positioned. In some embodiments, system 100 determines whether the sensor module is positioned to illuminate a subcutaneous blood vessel with light beam 132 (FIG. 3). If the sensor module is not so positioned, system 100 can alert the operator, e.g., with a visual or audio signal. The operator then adjusts the sensor module 102 with respect to the wrist. Alternatively, or in combination, the operator uses the system to change the location of the wrist to be illuminated by the light beam. In either case, the system can alert the operator with a signal when light beam 132 is positioned to illuminate a blood vessel. Once a selected spatial relationship between the light beam and blood vessel is achieved, the system illuminates the blood vessel with light and determines the in vivo blood property.

In some embodiments, system 100 selectively illuminates a blood vessel based on an automatically determined location of the blood vessel. The selective illumination may be automatic. For example, based on optical data obtained by sensor module 102, the processor 106 selects a location of the wrist 110 to be illuminated with a light beam. In various embodiments, the selected location is the skin 126 overlying a subcutaneous blood vessel (e.g., light beam 132 of FIG. 3) or offset from a subcutaneous blood vessel (e.g., light beam 132″ of FIGS. 6A and 6B). Processor 106 controls the system, e.g., light source 104 and/or sensor module 102, to selectively illuminate the location with the light beam. The processor determines the in vivo blood property based on detected light resulting from the selective illumination. For example, the selective illumination can allow the detection of light that has propagated each of at least two different distances from the illuminated portion of the blood vessel.

In some embodiments, the system determines the location of a blood vessel and the in vivo blood property from the same optical data. For example, the sensor module 102 may illuminate each of a plurality of discrete locations of the wrist and detect light resulting from the illumination of each discrete location. In general, the detected light resulting from the illumination of each discrete location can be distinguished, whether spatially or temporally, from the detected light resulting from the illumination of other locations. The processor determines the location of a subcutaneous blood vessel based on the detected light. Based on the relative positions of the illuminated locations with respect to the blood vessel, the processor determines whether the illumination of a particular one (or more) of the discrete locations resulted in the illumination of the blood vessel. If so, the system can determine the blood property based on light that was detected upon the illumination of the particular discrete location. Alternatively, or in combination, the system can illuminate the particular location one or more additional times and determine the in vivo property based on light detected upon the additional illuminations.

In some embodiments, system 100 determines a relative Hb concentration and/or Hct value in combination with or as an alternative to an absolute Hb concentration and/or Hct value. For example, system 100 can be used to monitor a subject's Hb or Hct at different points in time, as during a surgical procedure. As lost blood (e.g., blood lost through wounds or incisions) is replaced with plasma or other blood substitute lacking red blood cells, the subject's Hb or Hct values decrease relatively. System 100 can monitor such decrease (and any relative increase upon replenishing the red blood cell population) without necessarily determining the absolute Hb or Hct value. A medical practitioner can introduce fluids and/or red blood cells to the subject based on the relative Hb or Hct values.

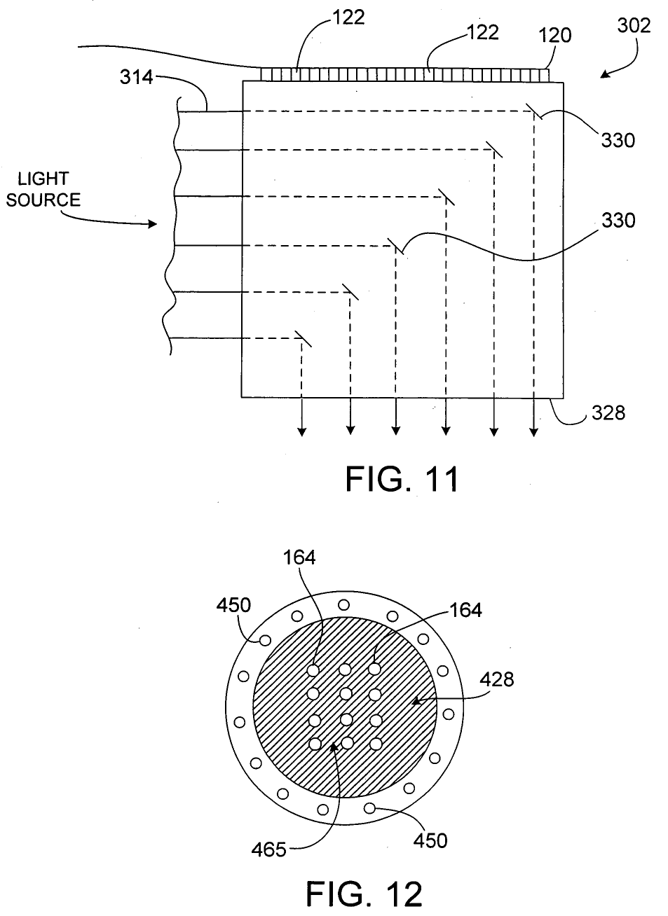

While optical fibers 114 have been described as extending to optical face 128 of sensor module 102, other configurations can be used. For example, referring to FIG. 11, a sensor module 302 includes a plurality of directional elements 314, e.g., micro-mirrors or prisms, configured to direct light from a light source from a side 330 of the sensor module toward an optical face 328. The directional elements 314 along a given row can be arranged in staircase fashion to direct light introduced along different paths through the sensor module toward optical face 328. A sensor module can include fibers to guide light to an interior of the sensor module and directional elements to direct the light to an optical face of the module. The fibers or light guides that guide light from a periphery of the sensor module to an interior of the sensor module can be spaced apart from the optical face of the module as in module 102 or can extend along the optical face itself. In some embodiments, light sources, e.g., LED's, are positioned to project light from the optical face without a fiber or directional element. For example, the light sources may be disposed within a sensor module.

While sensor module 102 has been described as including fibers 114 for illuminating skin 126, other illumination sources may be used. For example, referring to FIG. 12, a sensor module 402 includes an optical face 128 having a plurality of terminal fiber ends 164 for projecting light from the optical face. A region 465 of the optical face is configured to receive light and transmit the light to a detector. A plurality of light emitting elements 450, e.g., terminal optical fiber ends or light emitting diodes (LED's), surround the optical face 428. Light emitting elements 450 generally illuminate the subcutaneous tissue and vessels beneath optical face 428. Processor 106 can determine, e.g., a location of a blood vessel based on light detected upon illumination with elements 450. Processor 106 can then select a terminal end 164 to project a light beam into the blood vessel.

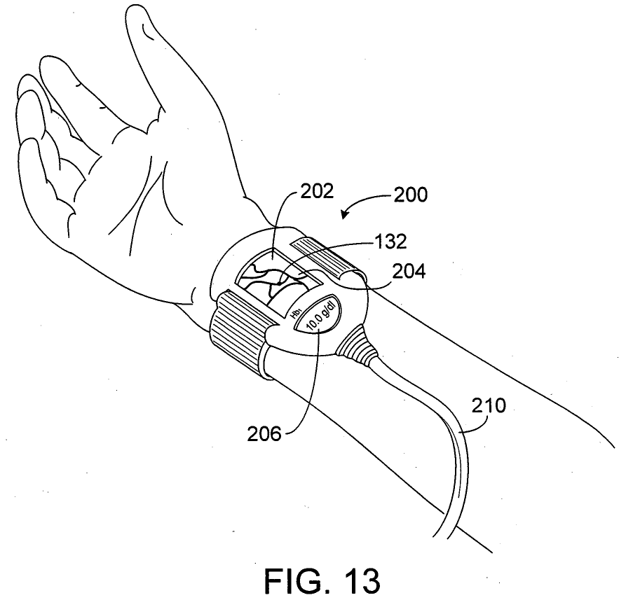

Referring to FIG. 13, an integrated system 200 determines an in vivo property of tissue or blood of a subject. System 200 includes a light source for illuminating skin and subcutaneous tissue of the subject. A multidimensional detector, e.g., a CCD, detects light resulting from the illumination and converts the detected light to optical data. A display, e.g., a liquid crystal display 202, displays the optical data as an image 204 including at least one subcutaneous blood vessel. The image can also include at least one light beam or a marker indicative of a location of the subject to be illuminated by a light beam. Hence, an operator can determine from the display whether the light beam overlaps a blood vessel. Alternatively, or in addition, the processor of the system 200 can automatically determine the location of the blood vessel and selectively illuminate the blood vessel with a light beam.

System 200 also includes an output, e.g., an output display 206 for output of the tissue or blood property, e.g., an Hct value. System 200 can be directly linked via a connector 210 or wirelessly linked to a power supply or processing module for monitoring the tissue or blood property along with other parameters. Connector 210 can include optical fibers for carrying light to or from the system 200. Hence, either or both the light source and detector can be positioned remote from the portion shown.

Once system 200 has been positioned to illuminate a blood vessel, the system can continuously or intermittently determine the tissue or blood property during, e.g., a surgical intervention or diagnostic procedure. An operator can verify at any time that the light beam is properly positioned to illuminate the blood vessel. System 200 can determine if proper positioning is lost and to notify the operator of such event.

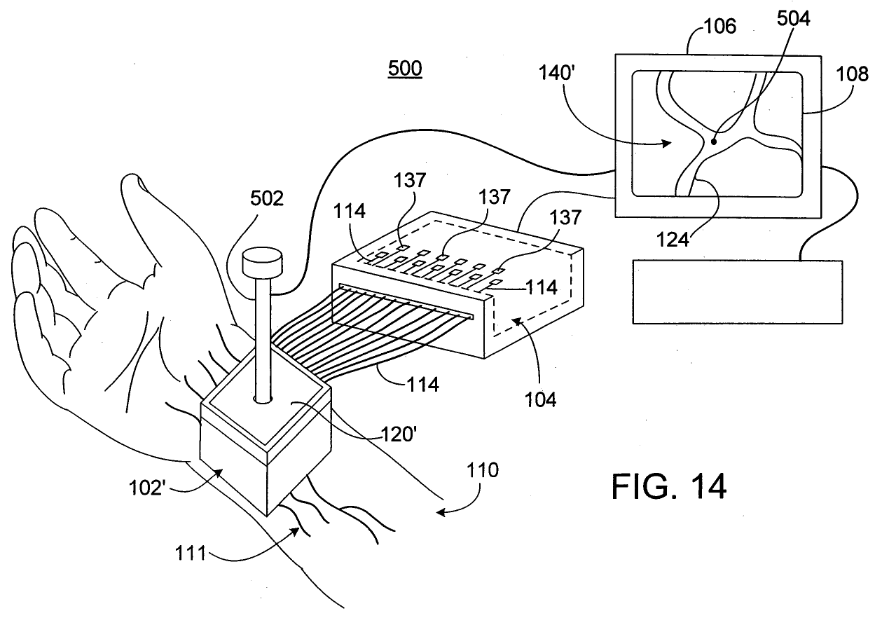

While systems for determining an in vivo blood property have been described, systems may perform additional or alternative functions. For example, referring to FIG. 14, a system includes a modified sensor module 102′ having an injection module 502 for performing an injection and/or marking the skin for later manipulation. When configured to perform an injection, module 502 automatically introduces or allows the manual introduction of a material, e.g., blood, saline solution, glucose solution, or medicine, for example, subcutaneously or intravenously, such as by injection via a target site into blood vessel 124. In marking mode, the module 502 may mark the skin, e.g., via ink, at the target site. System 500 can display an image 140′ indicative of a spatial relationship between an image of the target site 504 or location that will receive an injected material and one or more subcutaneous features, such as blood vessel 124. For example, the image 140′ can indicate whether the injection will be received within a blood vessel or offset from the blood vessel.

In some embodiments, an operator positions sensor module 102′ in an operative position with respect to a subject, e.g., with respect to skin of the subject, e.g., adjacent the wrist 110, contacting the skin of the wrist, or spaced apart from the wrist by coupling element 127. The operator manipulates the sensor module while observing the position of target site 504 and subcutaneous features. When a desired spatial relationship is achieved, the operator can manually or automatically inject a material via module 502. The module can include a needle or other injection device. System 500 can be configured to signal the operator when site 504 has a desired spatial relationship with a blood vessel or other subcutaneous feature. Rather than or in addition to injecting a material, the module may simply mark site 504 for later injection or manipulation. Although module 502 is shown oriented normal to the skin, other orientations, e.g., sub-ninety degree angles, with respect to the skin can be used.

Any of the methods discussed herein can be implemented in hardware or software, or a combination of both. The methods can be implemented in computer programs using standard programming techniques following the methods and figures described herein. Program code can be applied to input data, e.g., image data and/or data resulting from detected light, to perform the functions described herein and generate output information. The output information can be applied to one or more output devices such as display 108. Each program may be implemented in a high level procedural or object oriented programming language to communicate with processor 106, e.g., a computer system, handheld processing device, or the like. However, the programs can be implemented in assembly or machine language, if desired. In any case, the language can be a compiled or interpreted language. Moreover, the program can run on or be implemented by dedicated integrated circuits preprogrammed for that purpose.

Each such program can be stored on a storage medium or device (e.g., ROM, compact disk, or magnetic diskette) readable by a general or special purpose programmable processor. The program can also reside in a cache or a main memory during program execution. The analysis methods can also be implemented as a computer-readable or machine-readable storage medium, configured with a computer program, where the storage medium so configured causes a processor to operate in a specific and predefined manner to perform the functions described herein.

### OTHER EMBODIMENTS

In the embodiments shown, optical fibers 114 may be fixed with respect to optical face 128. In other embodiments, a sensor module moves, e.g., scans, a light beam with respect to a subject. A multidimensional detector detects light resulting from illumination with the beam. For example, the sensor module may move the beam by scanning the terminus of an optical fiber or by directing the beam with a movable optic, e.g., a positionable mirror.

System 100 is not limited to determinations of in vivo blood properties based on the ratio of two or more detected light intensities, whether corrected for contributions from skin and non-blood subcutaneous tissue or not.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining an in vivo blood property, the method comprising:
irradiating a first location of a subject's skin with incident light;
detecting exit light resulting from irradiating the first location, at least some of the exit light having passed through a blood vessel of the subject and exited the subject's skin at a distance from the first location;
irradiating a second location of the subject's skin with incident light;
detecting reference light resulting from irradiating the second, different location, at least some of the reference light having passed through at least some subsurface tissue of the subject without passing through the blood vessel; and
determining the in vivo blood property based on the first exit light and the reference light.

2. The method of claim 1, wherein:
the exit light is first exit light and the distance is a first distance; and
the method further comprises:
detecting second exit light resulting from irradiating the first location, at least some of the second exit light having passed through a blood vessel of the subject and exited the subject's skin at a second distance from the first location, the first and second distances being different,
wherein:
determining the in vivo blood property comprises determining the in vivo property based on the first and second exit light and the reference light.

3. The method of claim 2, wherein the first and second illumination locations are spaced apart from one another.

4. The method of claim 3, wherein:
detecting the first exit light comprises detecting light that has exited the skin through a first area of skin centered about the first distance;
detecting the second exit light comprises detecting light that has exited the skin through a second area of skin centered about the second distance;
detecting the reference light comprises detecting light that has exited the skin through a third area of skin that is larger than the first or second areas; and
determining the in vivo property comprises determining the in vivo property based on the reference light that has exited the skin through the third area of skin.

5. The method of claim 4, wherein the third area of the subject's skin is at least about 10 times larger than the first or second areas.

6. The method of claim 4, wherein:
the third area has a lateral dimension of at least about 2 mm;
the first area has a maximum lateral dimension of about 0.75 mm or less; and
the second area has a maximum lateral dimension of about 0.75 mm or less.

7. The method of claim 4, wherein irradiating the first location and irradiating the second, different location each comprise irradiating the skin with a beam of incident light having a diameter about the same as or less than a diameter of the blood vessel.

8. The method of claim 4, wherein determining the in vivo blood property based on the first and second exit light and the reference light comprises determining the in vivo blood property based on at least:
a first portion of the reference light indicative of the total amount of light exiting the skin through the third area;
a second portion of the reference light that is indicative of the total amount of light exiting the skin through a fourth area centered about the first distance from the second illumination location, wherein the size of the first and fourth areas are about the same; and
a third portion of the reference light that is indicative of the total amount of light exiting the skin through a fifth area centered about the second distance from the second illumination location, wherein the size of the second and fifth areas are about the same.

9. The method of claim 3, wherein the blood vessel has a diameter of at least about 500 microns.

10. The method of claim 8, wherein the reference light is detected without having passed through a blood vessel with a diameter greater than about 100 microns.

11. The method of claim 3, wherein detecting the first exit light comprises detecting light that has exited the subject's skin through an area that has a maximum dimension smaller than a difference between the first and second distances.

12. The method of claim 3, further comprising:
detecting second and third reference light resulting from irradiating the first location of the subject's skin with second incident light, the second incident light having a wavelength that is more attenuated by blood than a wavelength of the first incident light, the second reference light exiting the subject's skin at the first distance from the first location, the third reference light exiting the subject's skin at the second, different distance from the first location,
wherein,
determining the in vivo blood property comprises determining the in vivo blood property based on the first and second exit light and the first, second, and third reference light.

13. A method of determining an in vivo blood property, comprising:
detecting first exit light I_{1,1} resulting from irradiating a first location of a subject's skin with incident light, at least some of the first exit light having passed through a blood vessel of the subject and exited the subject's skin at a first distance from the first location,
detecting first reference light R_{1,1} resulting from irradiating the first location of the subject's skin with second incident light, at least some of the first reference light having passed through subsurface tissue of the subject and exited the subject's skin at the first distance from the first location, the second incident light having a wavelength that is more attenuated by blood than a wavelength of the first incident light,
detecting second reference light I_{2,T} resulting from irradiating a second, different location of the subject's skin with third incident light, at least some of the second reference light having passed through at least some subsurface tissue of the subject without passing through the blood vessel, wherein detecting the second reference light I_{2,T} comprises detecting light that has exited the subject's skin through an area of the subject's skin that is larger than an area through which either the first exit light or first reference light exited the subject's skin,
detecting third reference light I_{2,1} resulting from irradiating the second, different location of the subject's skin with incident light, at least some of the third reference light having passed through subsurface tissue of the subject without passing through the blood vessel and exited the subject's skin at the first distance from the second location,
determining the in vivo blood property based on the light I_{1,1, R}_{1,1}, I_{2,T}, I_{2,1}.

14. The method of claim 13, wherein determining the in vivo blood property comprises determining a first corrected light intensity I_{1,C} based at least in part on the relationship:
$${I}_{1 , C} = \frac{( {I}_{1 , 1} - {R}_{1 , 1} ) \times {I}_{2 , T}^{10}}{{I}_{2 , 1}}$$

15. The method of claim 13, comprising:
detecting second exit light I_{1,2} resulting from irradiating the first location of the subject's skin with first incident light, at least some of the second exit light having passed through the blood vessel of the subject and exited the subject's skin at a second distance from the first location,
detecting fourth reference light R_{1,2} resulting from irradiating the first location of the subject's skin with second incident light, at least some of the second light having passed through subsurface tissue of the subject and exited the subject's skin at the second distance from the first location,
detecting fifth reference light I_{2,2} resulting from irradiating the second, different location of the subject's skin with incident light, at least some of the fifth reference light having passed through subsurface tissue of the subject without passing through the blood vessel and exited the subject's skin at the second distance from the second location,
determining the in vivo blood property based on the light I_{1,1}, R_{1,1}, I_{2,T}, I_{2,1}, I_{1,2}, R_{1,2}, I_{2,2}.

16. The method of claim 13, wherein determining the in vivo blood property comprises determining a first corrected light intensity I_{1,C} based at least in part on the relationship:
$${I}_{1 , C} = \frac{( {I}_{1 , 2} - {R}_{1 , 2} ) \times {I}_{2 , T}}{{I}_{2 , 2}}$$
and determining a second corrected light intensity I_{2,C} based at least in part on the relationship:
$${I}_{2 , C} = \frac{( {I}_{1 , 1} - {R}_{1 , 1} ) \times {I}_{2 , T}}{{I}_{2 , 1}} .$$

17. A method, comprising:
automatically determining a location of a blood vessel of a subject;
illuminating the blood vessel with light by illuminating a first location of skin of the subject with incident light;
detecting exit light resulting from illuminating the first location of skin;
illuminating a second location of the skin of the subject with incident light, the second location being spaced apart from the first location;
detecting reference light resulting from illuminating the second location of skin, at least some of the reference light having passed through at least some sub-surface tissue of the subject without passing through the blood vessel; and
determining an in vivo blood property based on the exit light and the reference light.

18. The method of claim 17, wherein detecting the reference light comprises detecting light that has exited the subject's skin through an area of the skin that is larger than an area of the skin through which the exit light exited and determining the in vivo property comprises determining the in vivo property based on the reference light detected through the area of the subjects skin that is larger than the area through which the exit light exited the skin.

19. A system, comprising:
a light source configured to irradiate first and second spaced-apart locations of a subject's skin with incident light;
a detector configured to:
detect exit light resulting from irradiating the first location, at least some of the exit light having passed through a blood vessel of the subject and exited the subject's skin at a distance from the first location;
detect reference light resulting from irradiating the second, different location, at least some of the reference light having passed through at least some subsurface tissue of the subject without passing through the blood vessel; and
a processor configured to determine the in vivo blood property based on the first exit light and the reference light.

20. The system of claim 19, wherein the light source is configured to irradiate each of the first and second locations with a beam of incident light having a diameter of about 2 mm or less.
